(19) United States
(12) Patent Application Publication
(10) Pub. No.: US 2024/0122627 A1
(43) Pub. Date: Apr. 18, 2024

(54) SPINAL BONE FASTENER ASSEMBLY COMPRISING A PEDICLE SCREW WITH AT LEAST TWO BONE THREADS

(71) Applicants: HARALD KINZNER, CH-Zürich (CH); INTEGRUM MEDICAL AG, 8050 Zürich (CH)

(72) Inventors: Harald KINZNER, Zürich (CH); Rinaldo DE AGOSTINO, Zürich (CH); Tom OVERES, Langendorf (CH)

(73) Assignees: HARALD KINZNER, CH-Zürich (CH); INTEGRUM MEDICAL AG, 8050 Zürich (CH)

(21) Appl. No.: 18/266,460

(22) PCT Filed: Dec. 9, 2021
(86) PCT No.: PCT/IB2021/061532

§ 371 Date: Jun. 9, 2023

(30) Foreign Application Priority Data
Dec. 15, 2020 (CH) 01591/20

(51) Int. Cl. A61B 17/70; A61B 17/86

(52) U.S. Cl. CPC A61B 17/7032; A61B 17/8615; A61B 17/863; A61B 17/866

(57) ABSTRACT

A spinal bone fastener assembly (1) is proposed for stabilising the posterior spine. The assembly (1) comprises a pedicle screw (10), a rod receiving head (60), an insert (70) and a setscrew (80) intended to rigidly fixate a spinal rod (90). The pedicle screw (10) is longitudinally divided into a shaft tip section, a first shaft middle section, a second shaft middle section, a shaft transition section, a neck section, and a head section (21). The pedicle screw (10) comprises at least two parallel bone threads, namely a first bone thread and a second bone thread, both following a helical path around the shaft axis. The first and second bone threads at least in the second shaft middle section define a first substantially cylindrical thread section, and a second substantially cylindrical thread section, respectively. The first and second bone threads have different crest heights and/or outer thread diameters at least in the second shaft middle section.

### BACKGROUND OF THE INVENTION

### Field of the Invention

The present invention relates to a spinal bone fastener assembly, which may be part of a spinal posterior rod system. In one aspect of the invention, the spinal bone fastener assembly provides high stability thanks to its size and shape, and it further provides an efficient stress distribution at the implant-bone interface due to the elasticity of the materials used for the spinal bone fastener assembly.

### Discussion of Related Art

In orthopaedic surgeries around the spine, posterior spinal stabilisation systems are often placed to a target site to realign, correct and/or stabilise the spinal column to compensate for malalignment caused by for example degeneration of the spine, born malalignments, such as excessive lordosis, kyphosis and scoliosis, and for example trauma, such as fractures. A state-of-the-art pedicle screw assembly comprises a system of engaging elements that allows a surgeon to lock a rod, a rod receiving head and a pedicle screw simultaneously, by tightening a setscrew or a rod fastener against the rod in the rod receiving head. These elements form a bone fastener assembly. Prior to the locking step, the rod receiving head is movably connected to the pedicle screw head so that it is configured to swivel and rotate. The pedicle screw forms the interface with the vertebral body and provides the needed stability.

Placement of a construct, i.e., a bone fastener assembly, and correction of the spinal column requires application of high forces. Therefore, prior to the bony fusion of the spine, the bone fastener assembly, and more specifically the pedicle screws must withstand high loads and provide sufficient initial or primary stability. Primary stability is achieved by the size and shape of the screw as well as the form of the threads in relation to the target hole in which the screw is to be placed. For this purpose, many modern pedicle screws comprise a thicker section with multiple-lead threads (such as a double-lead or triple-lead thread) near the screw head. This section will increase the pressure on the bone and herewith the primary stability.

Over time, the bone will remodel or reshape around and against the pedicle screw and so it will provide a secondary stability. Important for a rapid and good quality of bone remodelling or reshaping is that the pedicle screw transfers the loads in a uniform manner. High and local peak-loads reduce the quality of the bone and can even cause loosening of a screw. As a result, the spinal stabilisation system will fail.

Another common cause of failure is the risk that the initial position of the system may be lost due to slippage between the pedicle screw head and the rod receiving head. Even worse, the single rod may not withstand the loads it is carrying over time, and a material-fatigue-related rod fracture may occur.

### SUMMARY OF THE INVENTION

It is an object of the present invention to overcome at least some of the problems associated with correcting the spinal column using a pedicle screw and rod constructs. A solution is needed that reduces the risks of a construct failure at the bone-implant interface, in particular due to material fatigue or connection slippage.

Therefore, there is a need for a bone fastener assembly that combines high primary stability with adequate insertion torques and optionally has material properties that enable an even distribution of loads. Moreover, the material properties of the screw and rod construct prevent failure due to fracture of the components.

According to a first aspect of the invention, there is provided a spinal bone fastener assembly as recited in the claims.

The proposed bone fastener assembly comprises a pedicle screw with a threaded shaft and a head. The threaded shaft has at least two parallel threads having different crest heights and/or thread diameters in a middle section of the thread. The at least two threads form at least a double-lead thread or double-start thread to enable fast implantation of the pedicle screw.

The pedicle screw may be made of an amorphous metal having a limit of elasticity which is at least 20 times higher than the elasticity limit of stainless steel. The higher elasticity provides a better division of the loads comparable to other implant materials, such as titanium, titanium alloys, or cobalt chromium alloys.

Other aspects of the invention are recited in the dependent claims attached hereto.

### BRIEF DESCRIPTION OF SEVERAL VIEWS OF THE DRAWINGS

Other features and advantages of the invention will become apparent from the following description of non-limiting example embodiments, with reference to the appended drawings, in which:

FIG. 1A depicts an example bone fastener assembly according to a first embodiment of the present invention;

FIG. 1B shows an exploded view of the bone fastener assembly shown in FIG. 1A;

FIG. 2A depicts an example pedicle screw in greater detail;

FIG. 2B depicts the pedicle screw in greater detail;

FIG. 2C depicts the pedicle screw in greater detail;

FIG. 2D depicts the pedicle screw in greater detail;

FIG. 2E depicts the pedicle screw in greater detail;

FIG. 2F depicts the pedicle screw in greater detail;

FIG. 2G depicts the pedicle screw in greater detail;

FIG. 2H depicts the pedicle screw in greater detail;

FIG. 2I depicts the pedicle screw in greater detail;

FIG. 2J depicts the pedicle screw in greater detail;

FIG. 3 depicts a variant of the pedicle screw;

FIG. 4 depicts the locking principle and further features of the bone fastener assembly in greater detail;

FIG. 5A depicts a setscrew in detail, and

FIG. 5B depicts an opposite side of the setscrew shown in FIG. 5A.

### DETAILED DESCRIPTION OF THE INVENTION

The embodiments of the present invention will now be described in detail with reference to the attached figures. The embodiments are described in the context of a posterior spinal construct comprising a pedicle-screw-based bone fastener assembly. Although the invention is specifically described in the context of stabilising the spine, the teachings of the invention are not limited to this environment. The teachings of the present invention are equally applicable to rod-based stabilisation constructs for other bones. When the words first and second are used to refer to different elements, it is to be understood that this does not necessarily imply or mean that the first and second elements are somehow structurally substantially different elements or that their dimensions are substantially different unless implicitly or explicitly stated. A bone fastener in this context means a structural element, which can be brought into the target bone, and forms a stable connection between the target bone and the remaining spinal construct. Most often, a bone fastener is a fastening element, such as a pedicle screw. Identical or corresponding functional and structural elements which appear in the different drawings are assigned the same reference numerals.

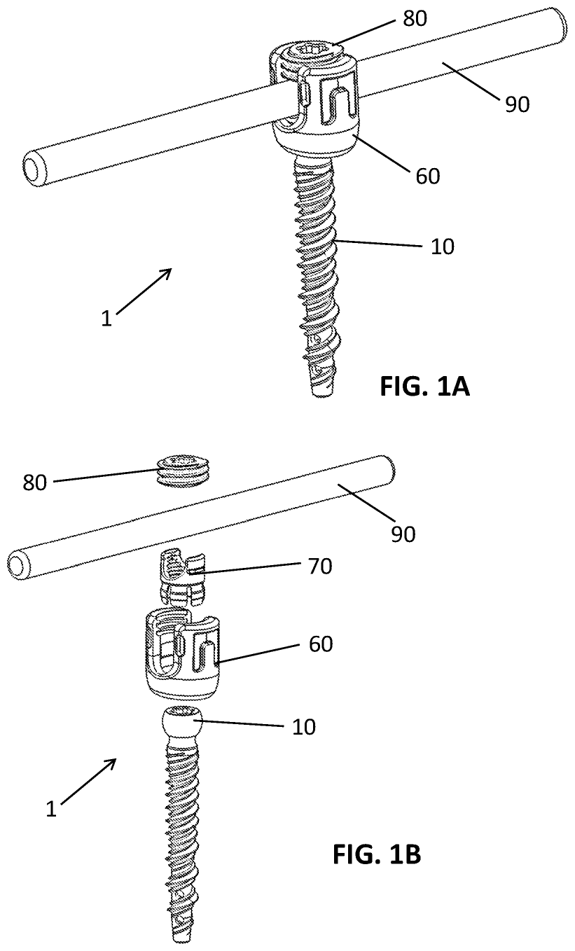

Referring to FIGS. 1A and 1B, a spinal bone fastener assembly 1 according to an example embodiment of the present invention is shown in perspective and exploded views, respectively. According to the present embodiment, the bone fastener assembly 1 comprises a pedicle screw 10, a rod receiving head 60, a locking insert 70, and at least one rod fastener, such as a setscrew 80. The bone fastener assembly 1 is configured to receive a spinal posterior stabilisation rod 90. As shows in FIG. 1A, the pedicle screw and the rod receiving head are in this example physically separate or independent elements. Furthermore, the rod receiving head is shaped and sized to receive the rod 90.

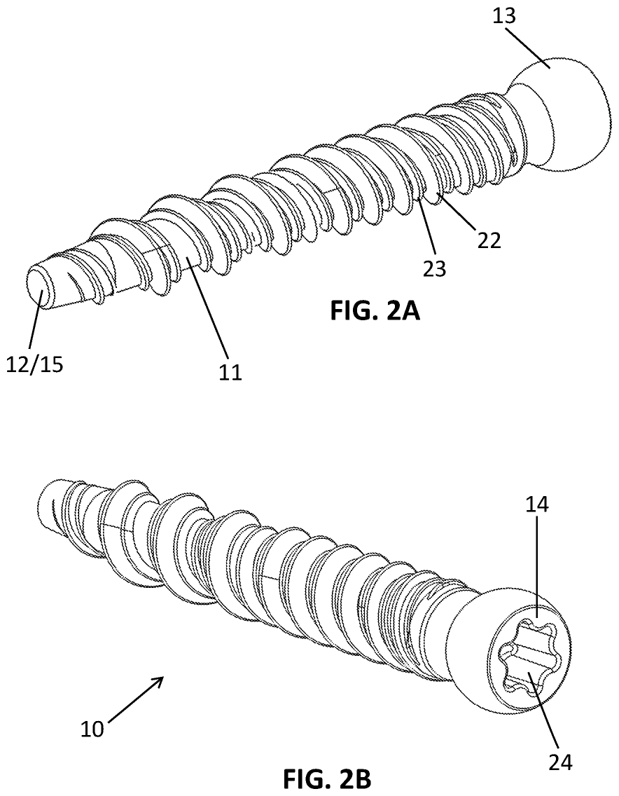

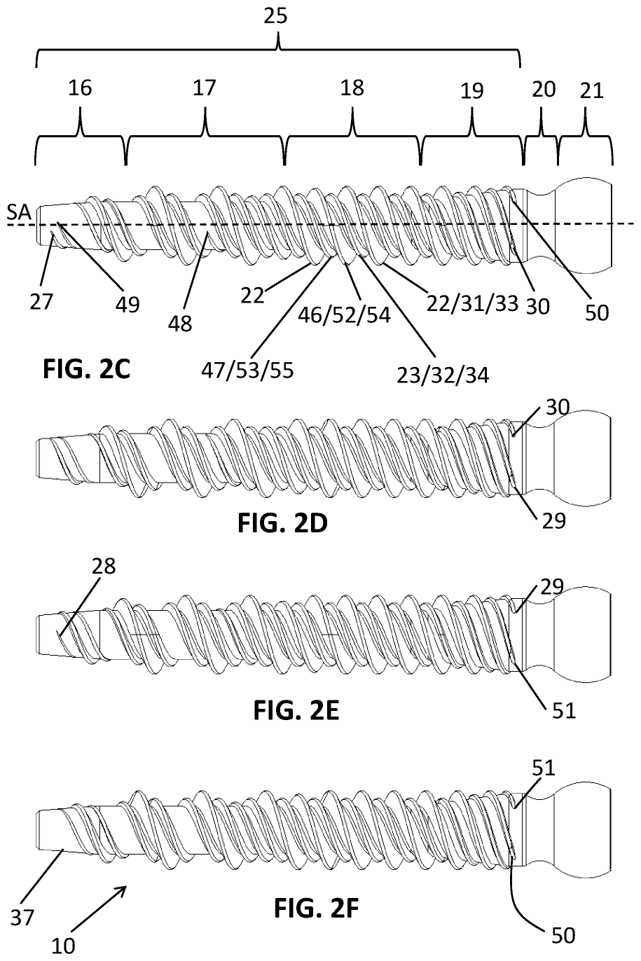

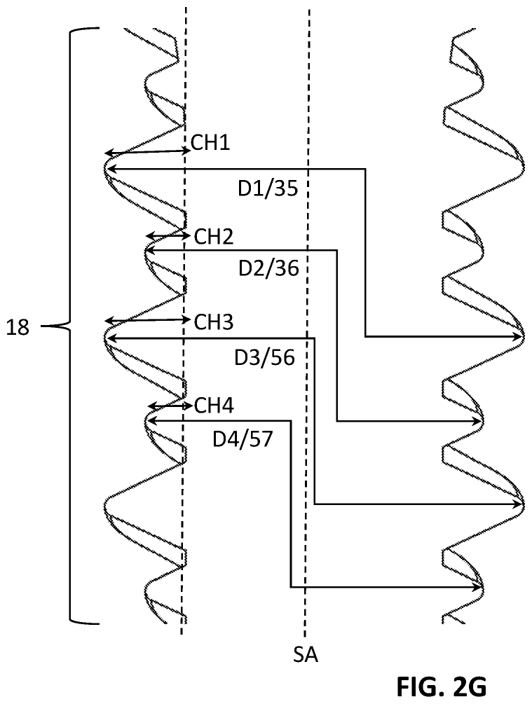

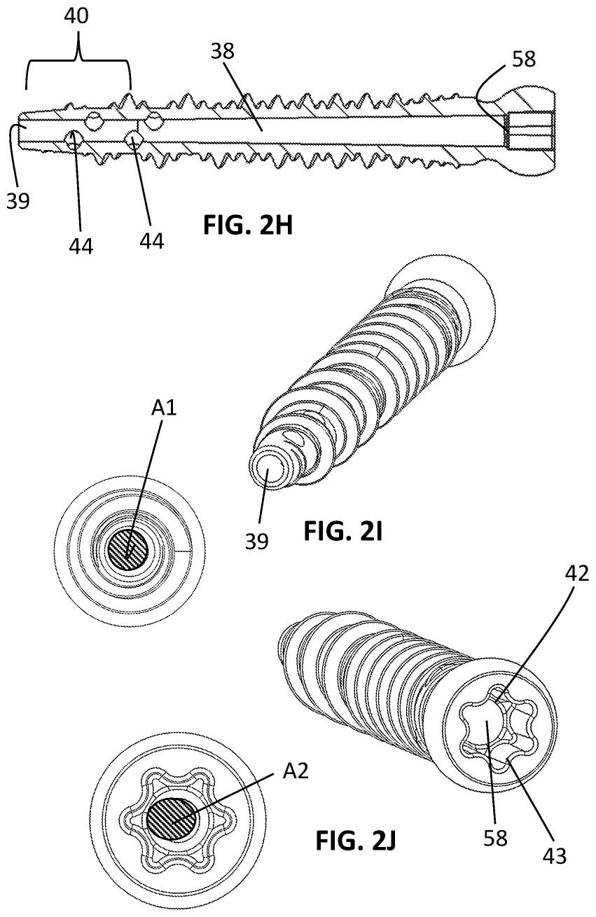

FIGS. 2A to 2J depict the pedicle screw 10 in greater detail. The pedicle screw 10 is shown in two perspective views in FIGS. 2A and 2B. The pedicle screw comprises an elongated shaft 11 extending between the pedicle screw tip 12 and a pedicle screw head 13. The head ends at the proximal end 14, and the tip ends at the distal end 15. For insertion, the head further comprises a drive 24 for engagement with an insertion tool, such as a screwdriver. The drive starts at the proximal end 14 and extends or protrudes into the pedicle screw head.

FIGS. 2C to 2F depict four views of the pedicle screw according to a third angle projection method. As depicted in FIG. 2C, the pedicle screw comprises, along the length of the pedicle screw from the distal end to the proximal end, a shaft tip section 16, a first shaft middle section 17, a second shaft middle section 18, a shaft transition section 19, a neck section 20 and a head section 21. As depicted, the pedicle screw head 13 is in this example a spherical head, and the shaft comprises at least two external or male threads 22, 23, thus forming a threaded shaft 25. The neck section forms a transition between the elongated shaft 11 and the pedicle screw head 13.

The shaft 11 comprises at least two parallel bone threads, namely a first bone thread 22, a second bone thread 23, a third bone thread 46 and a fourth bone thread 47, which follow a helical path along or around the shaft axis SA. The two or more bone threads form a multiple-lead thread to enable fast insertion of the pedicle screw. An exemplary lead length is 4 mm or greater for a pedicle screw with an average outer diameter OD of at least 6 mm. Lead length or simply lead is the linear travel the screw makes per one screw revolution. The pitch and lead are equal with single start screws. For multiple start screws the lead is the pitch multiplied by the number of starts. The bone threads have at least two distally arranged lead starts 27, 28, 48, 49 and at least two proximally arranged lead ends 29, 30, 50, 51. The bone threads form at least two separate thread helices, in this example a first thread helix 31, a second thread helix 32, a third thread helix 52, and a fourth thread helix 53 winding around the shaft axis, respectively having a first crest 33, a second crest 34, a third crest 54, and a fourth crest 55. The distance between each crest of two neighbouring thread helices is referred to as a pitch. In this example, the quadruple-lead thread defines a first, second, third and fourth pitch wherein any pitch may be equal or different in length.

As depicted, the lead starts 27, 28, 48, 49 and ends 29, 30, 50, 51 may gradually run out into the shaft or may end more abruptly. For example, the first bone thread 22 converges towards the shaft axis SA at the shaft tip section 16 and so gradually disappears. The second bone thread 23 converges towards the shaft axis in the middle section 17, 18 and also gradually disappears. In the present example, as depicted in FIG. 2G, each bone thread, and more specifically their respective crest, defines at least one imaginary substantially cylindrical thread section, namely a first, second, third and fourth cylindrical thread section 35, 36, 56, 57 along its helical path. The shaft axis SA coincides with the central axis of these imaginary cylinders. The cylindrical thread sections are predominantly arranged in the second middle shaft section 18. As depicted, the diameters of the first to fourth cylindrical sections are respectively defined by a first outer or major thread diameter D1, a second outer or major thread diameter D2, a third outer or major thread diameter D3, and a fourth outer or major thread diameter D4. It is to be noted that the translation distance along the shaft axis SA is not taken into consideration when defining the respective major thread diameter. In this case, the major diameter of a thread is the diameter of the imaginary co-axial cylinder that just touches the crest of an external thread. On the other hand, the minor or inner diameter is the diameter of an imaginary cylinder that just touches the roots of an external thread. Thus, a first crest height CH1, a second crest height CH2, a third crest height CH3, and a fourth crest height CH4 are defined as follows: CH1: (first major thread diameter D1−first minor thread diameter)/2; CH2: (second major thread diameter D2−second minor thread diameter)/2; CH3: (third major thread diameter D3−third minor thread diameter)/2; and CH4: (fourth major thread diameter D4−fourth minor thread diameter)/2. The crest heights are thus calculated from the thread roots to the respective crest as a projection distance (orthogonally to the thread axis SA). At least one outer thread diameter is greater than another outer thread diameter. In the present example, the first and third thread diameters are equally sized and are greater than the second and fourth thread diameters, which also are equally sized. Therefore, at least one bone thread has a different crest height measured from the same location along the pedicle screw length. In this example, the second shaft middle section is substantially cylindrically shaped and hence the crest heights of a given thread are substantially constantly sized.

In the present example, the shaft transition section 19 has a diverging shape. More specifically, the transition section has a tapered shape and therefore the crest heights reduce in height along the taper length TL towards the neck section 20 and the head section 21, while the shaft core diameter increases towards the neck section 20. The tapered shaft transition section will increase the pressure on the target bone when inserted into the bone and so increases the primary stability. For insertion and centralisation purposes, the tip section comprises a converging end 37. The converging end is configured as a conical or substantially conical tip, but it may also be configured as a curved or stepped tip.

FIG. 2H shows the pedicle screw in a cross-sectional view. The pedicle screw comprises a central cannulation 38 extending longitudinally through the pedicle screw. The cannulation is configured to receive a guide wire.

For example, in minimally invasive posterior stabilisation procedures, the screw trajectory is prepared using an awl or probe with a central channel. Before removal of the awl, a thin guide wire is placed into the awl channel. This guide wire marks the direction of the trajectory of the pedicle screw and the entry point into the bone. After removal of the awl, a pedicle screw or bone fastener assembly is placed over the guide wire and screwed into the target bone. Thanks to this technique, only little visualisation of the operation site is necessary. After placement of the implant, the guide wire is removed.

Typically, the pedicle screw cannulation is a cylindrical central channel extending through the pedicle screw between its proximal and distal ends 14, 15, and which is minimally oversized in relation to the guidewire. When a pedicle screw is placed over a guide wire, often tissue gets trapped between the cannulation wall and the guide wire. As a result, it may require greater forces to remove the guide wire after insertion of the pedicle screw. Another reason may be a small discrepancy between the direction of the guide wire and the direction of the final trajectory the pedicle screw is orienting itself during insertion.

In order to overcome this problem, according to one variant as depicted in FIG. 2H, the pedicle screw cannulation 38 comprises a cannulation entry 39 at the bone fastener tip or tip section 16 and a cannulation exit 58 at the head section 21. The entry and exit have a first cross-sectional area A1 and a second cross-sectional area A2, respectively. The first cross-sectional area is substantially constant over a first cannulation portion 40. The first cross-sectional area is smaller than the second cross-sectional area and is minimally oversized in relation to the guide wire it is intended to engage over. Hence, the cannulation will only provide guidance to the guide wire at the tip section. In the present example, the first cannulation portion 40 extends over a length of at least 2 mm. However, according to another example, the first cannulation portion has a length of at least 5 mm. The difference between the cross-sectional areas implicitly generates a transition section. In the present example, the transition section is configured as a substantially constant diverging taper. Alternatively, a stepped bore or a curved transition may serve the same purpose.

In one embodiment, the pedicle screw is configured to be manufactured by injection moulding, wherein a liquid metal is injected into a mould, and it is made to cure rapidly. The diverging taper provides a relief angle which is advantageous for this specific manufacturing method. To improve manufacturability, the pedicle screw may comprise rounded edges, such as inner rounded corners 42 and outer rounded edges 43 around the drive. Rounded corners and edges improve the flow of the liquid material during moulding and improve homogeneity of the final part.

As depicted, at the tip section 16 and/or first middle section 17, the pedicle screw may comprise one or more bores, channels, fenestrations or openings 44, which are open towards the outside of the pedicle screw. In some clinical cases, in a very osteoporotic bone, a pedicle screw cannot provide the needed primary stability. In these cases bone cement is injected into the target bone through the cannulation and the openings. The bone cement then cures around the pedicle screw providing extra stability. These fenestrations help create a homogeneous distribution of the bone cement around the pedicle screw as the bone cement can be spread around the pedicle screw through these fenestrations.

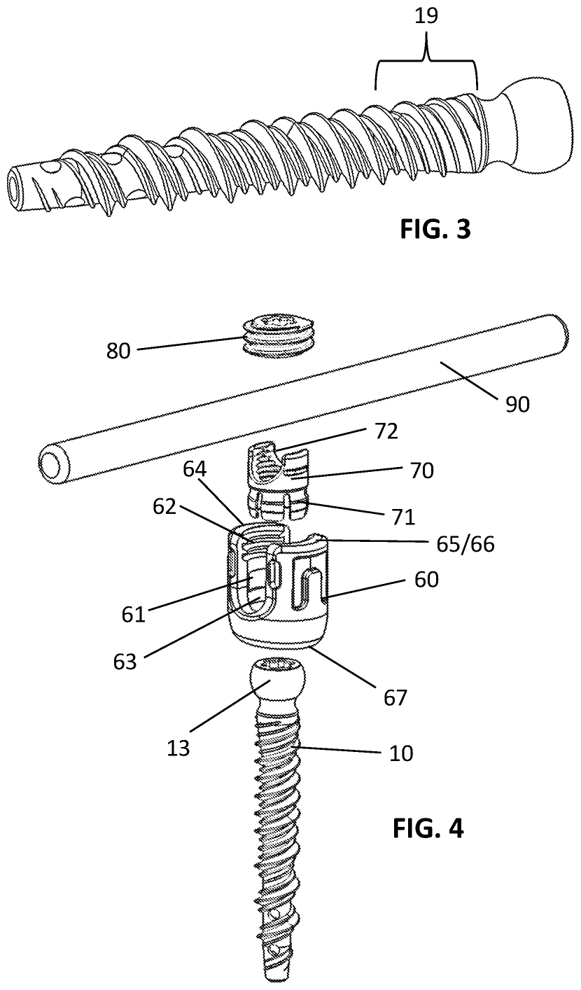

In the screw illustrated in FIGS. 2A to 2J, the helices remain prominent in relation to the transition section 19. Although the crest height is reduced, at least one of the helices is projecting or protruding from the shaft transition section. Referring to FIG. 3, an alternative design is shown, wherein the helices run out into the shaft transition section and fully disappear. Thus, in this example, the crests end flush with the transition section.

FIG. 4 shows the bone fastener assembly including a rod 90 in greater detail. The bone fastener assembly comprises the pedicle screw 10, the rod receiving head 60, the insert 70 and a setscrew 80. The rod is positioned between the setscrew and the insert. The rod receiving head 60 comprises a first passage 61 for receiving the rod, a thread feature 62 for receiving or engaging with the setscrew 80 and an inner lumen 63 for receiving the insert 70. At either side or both sides of the passage, a passage side wall 64, 65 is present. In this example, the insert is assembled into the rod receiving head from the proximal side 66 and is inhibited from passing through the head at the distal side 67. The insert 70 comprises flexible legs 71 which are sized and shaped to engage around the pedicle screw head 13 in a movement inhibiting clamping manner. The insert further comprises a second passage 72 for receiving the rod 90. The central axes of the first and second passages are substantially parallel to the longitudinal axis of the rod 90.

By means of tightening the setscrew, the rod is pressed down into the insert 70 whilst the rod receiving head 60 is pulled towards the rod 90. Simultaneously the insert is pressed down into the lumen 63 of the rod receiving head, and the lumen causes the legs of the insert to deflect inwards. Moreover, the legs simultaneously encompass the pedicle screw head and inhibit any motion thereof. As a result, the spinal bone fastener assembly is rigidly blocked.

For the full fixation of the bone fastener assembly including the rod, high torques are applied to the setscrew. In some designs, these forces even cause the passage sidewalls to deflect outwards. Such a deflection can make the setscrew disengage, and the construct would fail.

To prevent this disengagement, in one example, the rod receiving head is shaped elliptical, and thus having an oval-shaped outline, wherein the longest length is arranged substantially orthogonally to the passage central axis. Hence, the side walls are thicker and provide a greater bending resistance and therefore withstand greater forces.

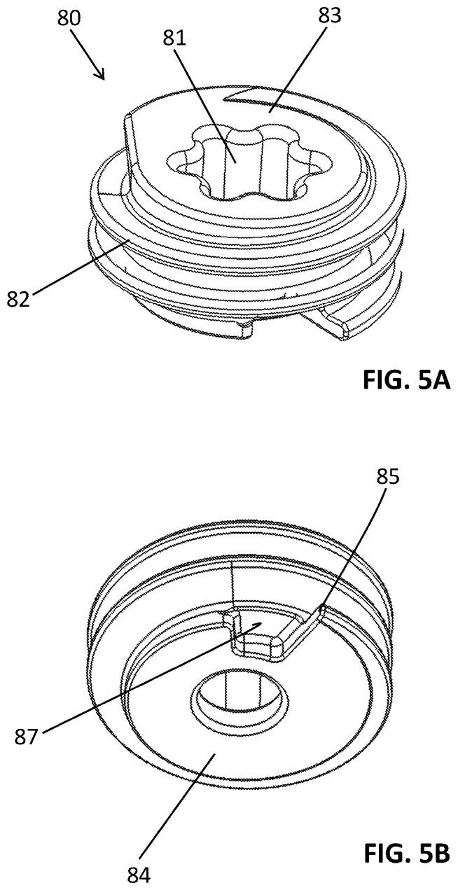

FIGS. 5A and 5B show an example setscrew 80 in detail. As described, the setscrew is configured for engagement, and in this case for threaded engagement, with the rod receiving head, and it is used to lock the full construct. Hence, the setscrew comprises a second drive 81 and an external thread 82 that is sized and shaped to engage into the rod receiving head. Moreover, the setscrew comprises a top side 83 and a bottom side 84. When engaged in the rod receiving head and against the rod 90, the bottom side faces the rod.

The setscrew is the last component to be placed. In some cases, prior to the placement of the setscrew, tissue or bone cement can get stuck in the thread feature of the rod receiving head. These foreign materials can compromise the final rigidity of the construct. Therefore, the setscrew may further comprise a cleaning feature, cleaning nose or cleaning surface 85 that captures and removes foreign materials or debris out of the thread feature. A pocket or recess 87 captures the debris. The pocket is in this example provided at the bottom side 84 of the setscrew 80, which is thus the rod facing surface when the bone screw assembly has been assembled.

Implants, such as spinal bone fastener assemblies, are commonly made of biocompatible materials, such as any one of titanium, titanium alloys, stainless steel, and cobalt chromium steel. According to the present embodiments, at least one element of the bone screw assembly and/or the accompanying rod may be made of a biocompatible amorphous metal. An amorphous metal, which is also known as metallic glass or glassy metal, is a solid metallic material, typically an alloy, with disordered atomic-scale structure. Most metals are crystalline in their solid state. This means they have a highly ordered arrangement of atoms. Amorphous metals are non-crystalline having a glass-like structure. But unlike common glasses, such as window glass, which are usually electrical insulators, amorphous metals have good electrical conductivity and they also display superconductivity at low temperatures. The amorphous metal may comprise the following first chemical composition and/or second chemical composition, the element concentration values below being given in weight percentages, namely the first chemical composition:
Zirconium (Zr): Balance;
Copper (Cu): 16% plus/minus 5%;
Nickel (Ni): 12% plus/minus 5%; and
Titanium (Ti): 3% plus 5%, minus 2%, and/or the second chemical composition:
Zirconium (Zr): Balance;
Copper (Cu): 24% plus/minus 5%;
Aluminium (Al): 4% plus 5%, minus 2%; and
Niob (Nb): 2% plus 5%, minus 1%.

In the above described example, the balance is the major chemical component or element. Depending on the exact percentage of the other components and possible minor impurities or other elements with a very minor weight percentage, the balance percentage gives the remaining percentage of the total 100%. The first and second compositions describe the four elements with the greatest weight percentage share.

According to an example, the amorphous metal has a limit of elasticity or elastic limit which is at least 20% higher in comparison to stainless steel. More preferably, the amorphous metal has a limit of elasticity which is at least 70% higher in comparison to stainless steel. By elastic limit is meant here the maximum stress or force per unit area within a solid material that can arise before the onset of permanent deformation. When stresses up to the elastic limit are removed, the material thus resumes its original size and shape. Stresses beyond the elastic limit cause a material to yield. The higher elasticity provides a better division of the loads comparable to other implant materials, such as titanium, titanium alloys, or cobalt chromium alloys. Amorphous metals allow for processing by means of injection moulding and additive manufacturing, i.e., three-dimensional printing. Therefore, at least one element of the bone screw assembly or the accompanying rod may be manufactured by means of injection moulding and/or additive manufacturing.

While the invention has been illustrated and described in detail in the drawings and foregoing description, such illustration and description are to be considered illustrative or exemplary and not restrictive, the invention being not limited to the disclosed embodiments. Other embodiments and variants are understood and can be achieved by those skilled in the art when carrying out the claimed invention, based on a study of the drawings, the disclosure and the appended claims. Further embodiments or variants may be obtained by combining any of the teachings above.

In the claims, the word “comprising” or “including” does not exclude other elements or steps, and the indefinite article “a” or “an” does not exclude a plurality. The mere fact that different features are recited in mutually different dependent claims does not indicate that a combination of these features cannot be advantageously used. Any reference signs in the claims should not be construed as limiting the scope of the invention.

1. A spinal bone fastener assembly (1) for stabilising the posterior spine, the assembly (1) comprising:
a pedicle screw (10), a rod receiving head (60), an insert (70), and a setscrew (80) for rigidly fixating a spinal rod (90) within the rod receiving head (60),
the pedicle screw (10) longitudinally comprising a shaft tip section (16), a first shaft middle section (17), a second shaft middle section (18), a shaft transition section (19), a neck section (20), and a head section (21) configured to be held by the insert (70) within the rod receiving head (60), the pedicle screw (10) comprising at least a first bone thread (22) with a first major thread diameter (D1), and a second bone thread (23) with a second major thread diameter (D2), the first and second bone threads (22, 23) running parallel to each other and following a helical path on an elongated shaft (11) of the pedicle screw (10) around a shaft axis (SA) of the pedicle screw (10),
wherein at least in the second shaft middle section (18) a first crest with a first crest height (CH1) of the first bone thread (22) defines a first imaginary cylindrical thread section, and a second crest with a second crest height (CH2) of the second bone thread (23) defines a second imaginary cylindrical thread section such that the first crest height (CH1) is different from the second crest height (CH2) and/or the first major thread diameter (D1) is different from the second major thread diameter (D2) at least in the second shaft middle section (18).

2. The spinal bone fastener assembly (1) according to claim 1, wherein the first major thread diameter (D1) is greater than the second major thread diameter (D2) at least in the second shaft middle section (18), and wherein the first bone thread (22) runs out into the elongated shaft (11) in the shaft tip section (16).

3. The spinal bone fastener assembly (1) according to claim 1, wherein the first major thread diameter (D1) is greater than the second major thread diameter (D2) at least in the second shaft middle section (18), and wherein the second bone thread (23) runs out into the elongated shaft (11) in the first shaft middle section (17).

4. The spinal bone fastener assembly (1) according to claim 1, wherein the pedicle screw (10) comprises a central cannulation (38) having a cannulation entry (39) with a first cross-sectional area (A1) at the shaft tip section (16), and a cannulation exit (58) with a second cross-sectional area (A2) at the head section (21), and wherein the first cross-sectional area (A1) is smaller than the second cross-sectional area (A2).

5. The spinal bone fastener assembly (1) according to claim 4, wherein the first cross-sectional area (A1) remains substantially constant over a first cannulation portion (40) having a length of at least 2 mm, or more specifically at least 5 mm.

6. The spinal bone fastener assembly (1) according to claim 1, wherein in the shaft transition section (19), a shaft core diameter increases towards the neck section (20).

7. The spinal bone fastener assembly (1) according to claim 1, wherein at least one of the pedicle screw (10), the rod receiving head (60), the insert (70), and the setscrew (80) is made of an amorphous metal.

8. The spinal bone fastener assembly (1) according to claim 1, wherein at least one of the pedicle screw (10), the rod receiving head (60), the insert (70), and the setscrew (80) is manufactured by injection moulding or additive manufacturing.

9. The spinal bone fastener assembly (1) according to claim 1, wherein the setscrew (80) comprises a cleaning feature (85, 87) or a cleaning nose (85) configured to remove foreign materials out of a thread feature of the rod receiving head (60).

10. The spinal bone fastener assembly (1) according to claim 9, wherein the cleaning feature (85, 87) comprises a cleaning surface (85) at a rod facing side of the setscrew (80), and a pocket (87) at the rod facing side of the setscrew (80) for collecting the foreign materials.

11. The spinal bone fastener assembly (1) according to claim 1, wherein the rod receiving head (60) has an oval-shaped overall outline substantially parallel to a central axis of a rod passage (72) of the insert (70).

12. The spinal bone fastener assembly (1) according to claim 1, wherein at least one of the pedicle screw (10), the rod receiving head (60), the insert (70), and the setscrew (80) has a chemical composition, given as a weight percentage, comprising:
Zirconium (Zr): balance;
Copper (Cu): 16% plus/minus 5%;
Nickel (Ni): 12% plus/minus 5%; and
Titanium (Ti): 3% plus 5%, minus 2%.

13. The spinal bone fastener assembly (1) according to claim 1, wherein at least one of the pedicle screw (10), the rod receiving head (60), the insert (70), and the setscrew (80) has a chemical composition, given as a weight percentage, comprising:
Zirconium (Zr): Balance;
Copper (Cu): 24% plus/minus 5%;
Aluminium (Al): 4% plus 5%, minus 2%; and
Niob (Nb): 2% plus 5%, minus 1%.

14. The spinal bone fastener assembly (1) according to claim 1, wherein the pedicle screw (10) further comprises a third bone thread (46) with a third major thread diameter (D3) and a third crest height (CH3), and running parallel to the first and a second bone threads (22, 23).

15. The spinal bone fastener assembly (1) according to claim 1, wherein the pedicle screw (10) further comprises a fourth bone thread (47) with a fourth major thread diameter (D4) and a fourth crest height (CH4), and running parallel to the first and a second bone threads (22, 23).

16. The spinal bone fastener assembly (1) according to claim 14, wherein the third major thread diameter (D3) equals the first major thread diameter (D1) and/or the third crest height (CH3) equals the first crest height (CH1) at least in the second shaft middle section (18), and/or the fourth major thread diameter (D4) equals the second major thread diameter (D2) and/or the fourth crest height (CH4) equals the second crest height (CH2) at least in the second shaft middle section (18).

17. The spinal bone fastener assembly (1) according to claim 1, wherein at least the shaft tip section (16) comprises one or more openings (44) leading to an internal channel (38) of the pedicle screw for injecting bone cement around the pedicle screw (10).
